(19) United States
(12) Patent Application Publication
(10) Pub. No.: US 2025/0030053 A1
(43) Pub. Date: Jan. 23, 2025

(54) ELECTROLYTIC SOLUTION FOR SECONDARY BATTERY, AND SECONDARY BATTERY

(71) Applicant: MURATA MANUFACTURING CO., LTD., Kyoto (JP)

(72) Inventor: Kentaro YOSHIMURA, Kyoto (JP)

(21) Appl. No.: 18/901,749

(22) Filed: Sep. 30, 2024

Related U.S. Application Data
(63) Continuation of application No. PCT/JP2023/021489, filed on Jun. 9, 2023.

(30) Foreign Application Priority Data
Jun. 23, 2022 (JP) 2022-101213

(51) Int. Cl. H01M 10/0568; C07C 69/606; H01M 4/58; H01M 4/583; H01M 10/0525

(52) U.S. Cl. CPC H01M 10/0568; C07C 69/606; H01M 4/5825; H01M 4/583; H01M 10/0525

(57) ABSTRACT

A secondary battery includes a positive electrode, a negative electrode, and an electrolytic solution. The electrolytic solution includes a triple bond compound and a fluorophosphoric acid salt. The triple bond compound includes a compound represented by Formula (1), a compound represented by Formula (2), or both. The fluorophosphoric acid salt includes a compound represented by Formula (3), a compound represented by Formula (4), or both.

### CROSS REFERENCE TO RELATED APPLICATIONS

The present application is a continuation of PCT patent application no. PCT/JP2023/021489, filed on Jun. 9, 2023, which claims priority to Japanese patent application no. 2022-101213, filed on Jun. 23, 2022, the entire contents of which are incorporated herein by reference.

### BACKGROUND

The present disclosure relates to an electrolytic solution for a secondary battery, and to a secondary battery.

Various kinds of electronic equipment, including mobile phones, have been widely used. Such widespread use has promoted development of a secondary battery as a power source that is smaller in size and lighter in weight and allows for a higher energy density. The secondary battery includes a positive electrode, a negative electrode, and an electrolytic solution (an electrolytic solution for the secondary battery). A configuration of the secondary battery has been considered in various ways.

Specifically, an electrolytic solution includes an oxygen-containing aliphatic compound that includes an alkynyl group including no active hydrogen or an alkynylene group including no active hydrogen. An electrolytic solution includes a compound having a carbon-carbon triple bond.

### SUMMARY

The present disclosure relates to an electrolytic solution for a secondary battery, and to a secondary battery.

Although consideration has been given in various ways regarding a configuration of a secondary battery, a battery characteristic of the secondary battery is not sufficient yet. Accordingly, there is room for improvement in terms of the battery characteristic of the secondary battery.

It is desirable to provide an electrolytic solution for a secondary battery, and a secondary battery each of which makes it possible to achieve a superior battery characteristic.

An electrolytic solution for a secondary battery according to an embodiment of the present technology includes a triple bond compound and a fluorophosphoric acid salt. The triple bond compound includes a compound represented by Formula (1), a compound represented by Formula (2), or both. The fluorophosphoric acid salt includes a compound represented by Formula (3), a compound represented by Formula (4), or both.

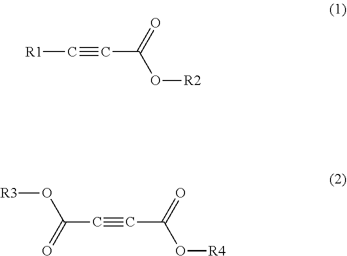

where each of R1 to R4 is an alkyl group.

M1PF₂O₂ (3)

where M1 is an alkali metal element.

M2₂PFO₃ (4)

where M2 is an alkali metal element.

A secondary battery according to an embodiment of the present technology includes a positive electrode, a negative electrode, and an electrolytic solution. The electrolytic solution has a configuration similar to the above-described configuration of the electrolytic solution for the secondary battery according to an embodiment of the present technology.

According to the electrolytic solution for the secondary battery of an embodiment of the present technology, or the secondary battery of an embodiment of the present technology, the electrolytic solution for the secondary battery includes the triple bond compound and the fluorophosphoric acid salt. Accordingly, it is possible to achieve a superior battery characteristic.

Note that effects of the present technology are not necessarily limited to those described herein and may include any of a series of effects in relation to the present technology.

### BRIEF DESCRIPTION OF THE FIGURES

FIG. 1 is a sectional diagram illustrating a configuration of a secondary battery according to an embodiment of the present technology.

FIG. 2 is a sectional diagram illustrating a configuration of a battery device illustrated in FIG. 1.

FIG. 3 is a sectional diagram illustrating a configuration of a secondary battery according to an embodiment.

FIG. 4 is a block diagram illustrating a configuration of an application example of the secondary battery according to an embodiment.

### DETAILED DESCRIPTION

The present technology is described below in further detail including with reference to the drawings according to an embodiment.

A description is given first of an electrolytic solution for a secondary battery (hereinafter simply referred to as an “electrolytic solution”) according to an embodiment of the present technology.

The electrolytic solution described here is a liquid electrolyte to be used in a secondary battery, which is an electrochemical device. However, the electrolytic solution may be used in other electrochemical devices besides the secondary battery. Specific examples of the other electrochemical devices include a capacitor.

The electrolytic solution includes any one or more of triple bond compounds, and the triple bond compounds are each a compound having a carbon-carbon triple bond (—C═C—).

For example, the triple bond compound includes a compound represented by Formula (1), a compound represented by Formula (2), or both. In other words, the triple bond compound may include only either the compound represented by Formula (1) or the compound represented by Formula (2), or may include both the compound represented by Formula (1) and the compound represented by Formula (2).

Hereinafter, the compound represented by Formula (1) is referred to as a “first triple bond compound”, and the compound represented by Formula (2) is referred to as a “second triple bond compound”.

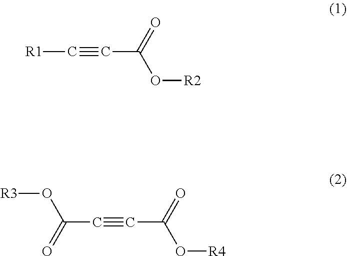

where each of R1 to R4 is an alkyl group.

A reason why the electrolytic solution includes the triple bond compound is that, even if a secondary battery including the electrolytic solution is repeatedly charged and discharged, a decrease in a discharge capacity is reduced.

In more detail, a synergistic action between the triple bond compound and a fluorophosphoric acid salt to be described later allows for formation of a film having superior electrochemical stability on a surface of a negative electrode 22, which improves electrochemical durability of the film. As will be described later, the film is formed on the surface of the negative electrode 22 through a stabilization process (an initial charge and discharge process) on the secondary battery after being assembled. This suppresses a decomposition reaction of the electrolytic solution on the surface of the negative electrode 22 even upon repeated charging and discharging, which reduces a decrease in the discharge capacity. In this case, a decomposition product of the film is prevented from easily seeping into the electrolytic solution upon charging and discharging.

Note that the film described above may be formed not only on the surface of the negative electrode 22 but also on a surface of a positive electrode 21. Accordingly, even upon repeated charging and discharging, a decomposition reaction of the electrolytic solution on the surface of the positive electrode 21 is suppressed, and corrosion of the positive electrode 21 is also suppressed.

As is apparent from Formula (1), the first triple bond compound is a compound including one carbonic acid ester group (—C(═O)—O—R2) together with the carbon-carbon triple bond.

Each of R1 and R2 is not particularly limited in kind as long as each of R1 and R2 is an alkyl group as described above. In this case, respective kinds of R1 and R2 may be the same as each other, or may be different from each other.

Note that carbon number of the alkyl group is not particularly limited. The alkyl group may have a straight-chain structure, or may have a branched structure.

Specific examples of the alkyl group include a methyl group, an ethyl group, a propyl group, a butyl group, a pentyl group, a hexyl group, a heptyl group, an octyl group, a nonyl group, and a decyl group.

The carbon number of the alkyl group is preferably within a range from 1 to 7 both inclusive, in particular. A reason for this is that the carbon number of the alkyl group is not too large, which allows for an improvement in solubility and compatibility of the first triple bond compound.

As is apparent from Formula (2), the second triple bond compound is a compound including two carbonic acid ester groups (—C(═O)—O—R3 and —C(═O)—R4) together with the carbon-carbon triple bond.

Details of each of R3 and R4 are similar to those of each of R1 and R2. Details of the alkyl group are as described above. That is, carbon number of the alkyl group is not particularly limited, and is preferably within a range from 1 to 7 both inclusive, in particular. A reason for this is that the carbon number of the alkyl group is not too large, which allows for an improvement in solubility and compatibility of the second triple bond compound.

Specific examples of the triple bond compound are as described below. Note that the specific examples of the triple bond compound described below are merely examples, and the specific examples of the triple bond compound may include a compound other than compounds described below.

Specific examples of the first triple bond compound include respective compounds represented by Formulae (1-1) to (1-10).

In more detail, the compound represented by Formula (1-1) is methyl 2-octynoate. The compound represented by Formula (1-2) is methyl 2-nonynoate. The compound represented by Formula (1-3) is methyl 2-hexynoate. The compound represented by Formula (1-4) is methyl 2-heptynoate. The compound represented by Formula (1-5) is ethyl 2-pentynoate. The compound represented by Formula (1-6) is ethyl 2-butynoate. The compound represented by Formula (1-7) is methyl 2-decynoate. The compound represented by Formula (1-8) is methyl 2-butynoate. The compound represented by Formula (1-9) is propyl 2-heptynoate. The compound represented by Formula (1-10) is butyl 2-heptynoate.

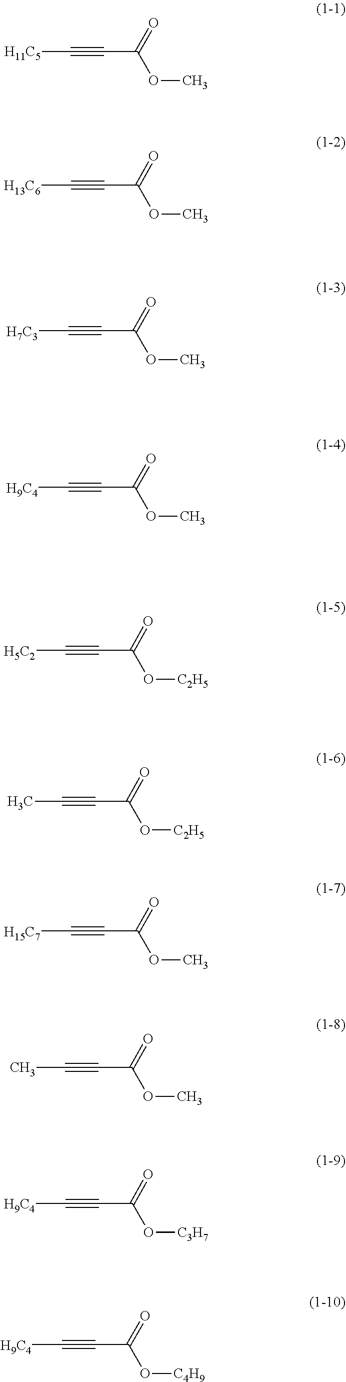

Specific examples of the second triple bond compound include respective compounds represented by Formulae (2-1) to (2-8). In more detail, the compound represented by Formula (2-1) is diisopropyl 2-butynedioate. The compound represented by Formula (2-2) is dipropyl 2-butynedioate. The compound represented by Formula (2-3) is dibutyl 2-butynedioate. The compound represented by Formula (2-4) is dipentyl 2-butynedioate. The compound represented by Formula (2-5) is dihexyl 2-butynedioate. The compound represented by Formula (2-6) is bis(2,2-dimethylpropyl) 2-butynedioate. The compound represented by Formula (2-7) is dioctyl 2-butynedioate. The compound represented by Formula (2-8) is bis(2-ethylhexyl) 2-butynedioate.

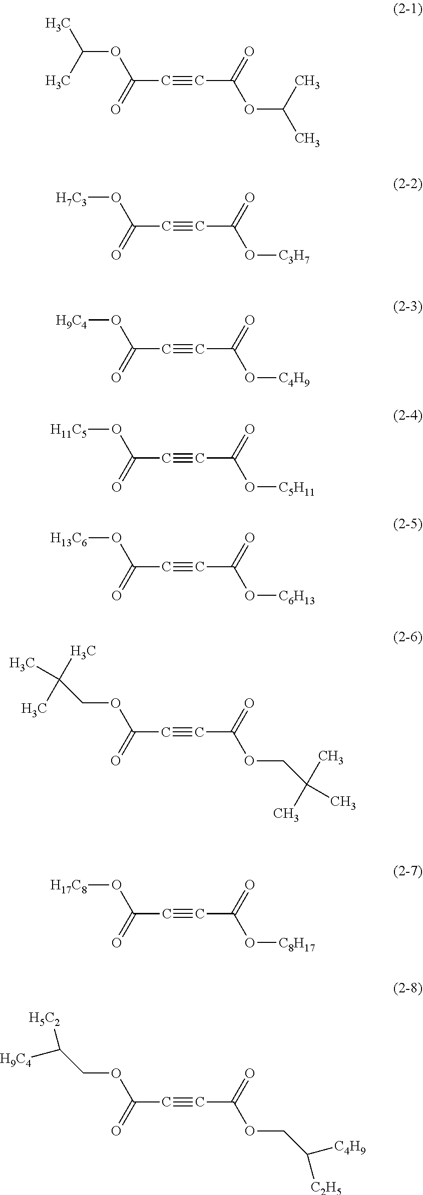

A content of the triple bond compound in the electrolytic solution is not particularly limited, and is preferably within a range from 0.01 wt % to 5 wt % both inclusive, in particular. A reason for this is that the electrochemical durability of the film provided on the surface of the negative electrode 22 sufficiently improves.

When the electrolytic solution includes both the first triple bond compound and the second triple bond compound, the content of the triple bond compound in the electrolytic solution described above is a sum of a content of the first triple bond compound in the electrolytic solution and a content of the second triple bond compound in the electrolytic solution.

When measuring the content of the triple bond compound, the electrolytic solution is analyzed to thereby calculate the content of the triple bond compound. When using a secondary battery including the electrolytic solution, the secondary battery is disassembled to thereby collect the electrolytic solution.

A method of analyzing the electrolytic solution is not particularly limited, and specifically includes any one or more of methods including, without limitation, inductively coupled plasma (ICP) optical emission spectroscopy, nuclear magnetic resonance (NMR) spectroscopy, and gas chromatography mass spectrometry (GC-MS).

The electrolytic solution includes any one or more of fluorophosphoric acid salts. The fluorophosphoric acid salts are each a salt including fluorine (F), phosphorus (P), and oxygen (O) as constituent elements.

For example, the fluorophosphoric acid salt includes a compound represented by Formula (3), a compound represented by Formula (4), or both. In other words, the fluorophosphoric acid salt may include only either the compound represented by Formula (3) or the compound represented by Formula (4), or may include both the compound represented by Formula (3) and the compound represented by Formula (4).

Hereinafter, the compound represented by Formula (3) is referred to as a “first fluorophosphoric acid salt”, and the compound represented by Formula (4) is referred to as a “second fluorophosphoric acid salt”.

M1PF₂O₂ (3)

where M1 is an alkali metal element.

M2₂PFO₃ (4)

where M2 is an alkali metal element.

A reason why the electrolytic solution includes the fluorophosphoric acid salt is that the synergistic action between the triple bond compound and the fluorophosphoric acid salt improves the electrochemical durability of the film provided on the surface of the negative electrode 22 as described above. This suppresses the decomposition reaction of the electrolytic solution on the surface of the negative electrode 22 even upon repeated charging and discharging, which reduces a decrease in the discharge capacity.

The fluorophosphoric acid salt serves to form the film on the surface of the negative electrode 22 as described above, and may also serve as an electrolyte salt to be described later.

As is apparent from Formula (3), the first fluorophosphoric acid salt is what is called a difluorophosphoric acid salt.

M1 is not particularly limited in kind as long as M1 is an alkali metal element as described above. Specific examples of the alkali metal element include lithium, sodium, and potassium.

In particular, the alkali metal element is preferably lithium. A reason for this is that, when the secondary battery is a lithium-ion secondary battery, the first fluorophosphoric acid salt also sufficiently serves as an electrolyte salt.

As is apparent from Formula (4), the second fluorophosphoric acid salt is what is called a monofluorophosphoric acid salt.

Details of M2 are similar to those of M1 described above. That is, the alkali metal element is not particularly limited in kind, and is preferably lithium in particular. A reason for this is that, when the secondary battery is a lithium-ion secondary battery, the second fluorophosphoric acid salt also sufficiently serves as an electrolyte salt.

Specific examples of the fluorophosphoric acid salt are as described below. Note that the specific examples of the fluorophosphoric acid salt described below are merely examples, and the specific examples of the fluorophosphoric acid salt may include a compound other than compounds described below.

Specific examples of the first fluorophosphoric acid salt include lithium difluorophosphate, sodium difluorophosphate, and potassium difluorophosphate.

Specific examples of the second fluorophosphoric acid salt include dilithium monofluorophosphate, disodium monofluorophosphate, and dipotassium monofluorophosphate.

A content of the fluorophosphoric acid salt in the electrolytic solution is not particularly limited, and is preferably within a range from 0.01 wt % to 2 wt % both inclusive, in particular. A reason for this is that the electrochemical durability of the film provided on the surface of the negative electrode 22 sufficiently improves.

When the electrolytic solution includes both the first fluorophosphoric acid salt and the second fluorophosphoric acid salt, the content of the fluorophosphoric acid salt in the electrolytic solution described above is a sum of a content of the first fluorophosphoric acid salt in the electrolytic solution and a content of the second fluorophosphoric acid salt in the electrolytic solution.

Details of a procedure for measuring the content of the fluorophosphoric acid salt are similar to details of the procedure for measuring the content of the triple bond compound described above.

Note that the electrolytic solution may further include a solvent. The solvent includes any one or more of non-aqueous solvents (organic solvents), and the electrolytic solution including the one or more non-aqueous solvents is what is called a non-aqueous electrolytic solution.

The non-aqueous solvent is, for example, an ester or an ether, more specifically, a carbonic-acid-ester-based compound, a carboxylic-acid-ester-based compound, or a lactone-based compound, for example.

The carbonic-acid-ester-based compound is, for example, a cyclic carbonic acid ester or a chain carbonic acid ester. Specific examples of the cyclic carbonic acid ester include ethylene carbonate and propylene carbonate. Specific examples of the chain carbonic acid ester include dimethyl carbonate, diethyl carbonate, and ethyl methyl carbonate.

The carboxylic-acid-ester-based compound is, for example, a chain carboxylic acid ester. Specific examples of the chain carboxylic acid ester include methyl acetate, ethyl acetate, methyl propionate, ethyl propionate, propyl propionate, ethyl trimethylacetate, methyl butyrate, and ethyl butyrate.

The lactone-based compound is, for example, a lactone. Specific examples of the lactone include γ-butyrolactone and γ-valerolactone.

Note that the ether may be, for example, 1,2-dimethoxyethane, tetrahydrofuran, 1,3-dioxolane, or 1,4-dioxane.

In addition, the electrolytic solution may further include an electrolyte salt. The electrolyte salt is a light metal salt such as a lithium salt.

Specific examples of the lithium salt include lithium hexafluorophosphate (LiPF₆), lithium tetrafluoroborate (LiBF₄), lithium trifluoromethanesulfonate (LiCF₃SO₃), lithium bis(fluorosulfonyl)imide (LiN(FSO₂)₂), lithium bis(trifluoromethanesulfonyl)imide (LiN(CF₃SO₂)₂), lithium tris(trifluoromethanesulfonyl) methide (LiC(CF₃SO₂)₃), lithium bis(oxalato) borate (LiB(C₂O₄)₂), lithium difluorooxalatoborate (LiBF₂(C₂O₄)), lithium difluorodi (oxalato) borate (LiPF₂(C₂O₄)₂), lithium tetrafluorooxalatophosphate (LiPF₄(C₂O₄)), lithium monofluorophosphate (LizPFO₃), and lithium difluorophosphate (LiPF₂O₂).

A content of the electrolyte salt is not particularly limited, and is specifically within a range from 0.3 mol/kg to 3.0 mol/kg both inclusive with respect to the solvent. A reason for this is that high ion conductivity is obtainable.

Note that the electrolytic solution may further include any one or more of additives.

For example, the one or more additives include any one or more of an unsaturated cyclic carbonic acid ester, a fluorinated cyclic carbonic acid ester, or a cyanated cyclic carbonic acid ester. A reason for this is that electrochemical stability of the electrolytic solution improves. This further suppresses the decomposition reaction of the electrolytic solution upon charging and discharging of the secondary battery, which further reduces a decrease in the discharge capacity even upon repeated charging and discharging.

The unsaturated cyclic carbonic acid ester is a cyclic carbonic acid ester having an unsaturated carbon bond (a carbon-carbon double bond). The number of unsaturated carbon bonds is not particularly limited, and may be only one, or two or more.

The unsaturated cyclic carbonic acid ester includes any one or more of a vinylene-carbonate-based compound, a vinyl-ethylene-carbonate-based compound, or a methylene-ethylene-carbonate-based compound.

The vinylene-carbonate-based compound is an unsaturated cyclic carbonic acid ester having a structure of a vinylene carbonate type. Specific examples of the vinylene-carbonate-based compound include vinylene carbonate (1,3-dioxol-2-one), methyl vinylene carbonate (4-methyl-1,3-dioxol-2-one), ethyl vinylene carbonate (4-ethyl-1,3-dioxol-2-one), 4,5-dimethyl-1,3-dioxol-2-one, 4,5-diethyl-1,3-dioxol-2-one, 4-fluoro-1,3-dioxol-2-one, and 4-trifluoromethyl-1,3-dioxol-2-one.

The vinyl-ethylene-carbonate-based compound is an unsaturated cyclic carbonic acid ester having a structure of a vinyl ethylene carbonate type. Specific examples of the vinyl-ethylene-carbonate-based compound include vinyl ethylene carbonate (4-vinyl-1,3-dioxolane-2-one), 4-methyl-4-vinyl-1,3-dioxolane-2-one, 4-ethyl-4-vinyl-1,3-dioxolane-2-one, 4-n-propyl-4-vinyl-1,3-dioxolane-2-one, 5-methyl-4-vinyl-1,3-dioxolane-2-one, 4,4-divinyl-1,3-dioxolane-2-one, and 4,5-divinyl-1,3-dioxolane-2-one.

The methylene-ethylene-carbonate-based compound is an unsaturated cyclic carbonic acid ester having a structure of a methylene ethylene carbonate type. Specific examples of the methylene-ethylene-carbonate-based compound include methylene ethylene carbonate (4-methylene-1,3-dioxolane-2-one), 4,4-dimethyl-5-methylene-1,3-dioxolane-2-one, and 4,4-diethyl-5-methylene-1,3-dioxolane-2-one. Here, a compound including only one methylene group is given as an example of the methylene-ethylene-carbonate-based compound; however, the methylene-ethylene-carbonate-based compound may include two or more methylene groups.

Note that the cyclic carbonic acid ester having an unsaturated carbon bond belongs to neither the fluorinated cyclic carbonic acid ester nor the cyanated cyclic carbonic acid ester, but belongs to the unsaturated cyclic carbonic acid ester.

The fluorinated cyclic carbonic acid ester is a cyclic carbonic acid ester including fluorine as a constituent element. The number of fluorine atoms is not particularly limited and may be only one, or two or more. That is, the fluorinated cyclic carbonic acid ester is a compound resulting from substituting one or more hydrogen atoms of the cyclic carbonic acid ester with one or more fluorine atoms.

Specific examples of the fluorinated cyclic carbonic acid ester include fluoroethylene carbonate (4-fluoro-1,3-dioxolane-2-one) and difluoroethylene carbonate (4,5-difluoro-1,3-dioxolane-2-one).

Note that the cyclic carbonic acid ester including fluorine as a constituent element belongs to neither the unsaturated cyclic carbonic acid ester nor the cyanated cyclic carbonic acid ester, but belongs to the fluorinated cyclic carbonic acid ester.

The cyanated cyclic carbonic acid ester is a cyclic carbonic acid ester including a cyano group. The number of cyano groups is not particularly limited and may be only one, or two or more. That is, the cyanated cyclic carbonic acid ester is a compound resulting from substituting one or more hydrogen atoms of the cyclic carbonic acid ester with one or more cyano groups.

Specific examples of the cyanated cyclic carbonic acid ester include cyanoethylene carbonate (4-cyano-1,3-dioxolane-2-one) and dicyanoethylene carbonate (4,5-dicyano-1,3-dioxolane-2-one).

Note that the cyclic carbonic acid ester including a cyano group belongs to neither the unsaturated cyclic carbonic acid ester nor the fluorinated cyclic carbonic acid ester, but belongs to the cyanated cyclic carbonic acid ester.

Further, the one or more additives include any one or more of a sulfonic acid ester, a sulfuric acid ester, a sulfurous acid ester, a dicarboxylic acid anhydride, a disulfonic acid anhydride, a sulfonic acid carboxylic acid anhydride, or a sulfobenzoic acid imide. A reason for this is that the electrochemical stability of the electrolytic solution improves. This further suppresses the decomposition reaction of the electrolytic solution upon charging and discharging, which further reduces a decrease in the discharge capacity even upon repeated charging and discharging.

Specific examples of the sulfonic acid ester include 1,3-propane sultone, 1-propene-1,3-sultone, 1,4-butane sultone, 2,4-butane sultone, and methanesulfonate propargyl ester.

Specific examples of the sulfuric acid ester include 1,3,2-dioxathiolane 2,2-dioxide, 1,3,2-dioxathiane 2,2-dioxide, and 4-methylsulfonyloxymethyl-2,2-dioxo-1,3,2-dioxathiolane.

Specific examples of the sulfurous acid ester include 1,3-propane sultone, 1-propene-1,3-sultone, 1,4-butane sultone, 2,4-butane sultone, and methanesulfonate propargyl ester. Specific examples of the sulfurous acid ester include 1,3,2-dioxathiolane 2-oxide and 4-methyl-1,3,2-dioxathiolane 2-oxide.

Specific examples of the dicarboxylic acid anhydride include 1,4-dioxane-2,6-dione, succinic anhydride, and glutaric anhydride.

Specific examples of the disulfonic acid anhydride include 1,2-ethanedisulfonic anhydride, 1,3-propanedisulfonic anhydride, and hexafluoro 1,3-propanedisulfonic anhydride.

Specific examples of the sulfonic acid carboxylic acid anhydride include 2-sulfobenzoic anhydride and 2,2-dioxooxathiolane-5-one.

Specific examples of the sulfobenzoic acid imide include o-sulfobenzimide and N-methylsaccharin.

Further, the one or more additives include any one or more of nitrile compounds. A reason for this is that the electrochemical stability of the electrolytic solution improves. This further suppresses the decomposition reaction of the electrolytic solution upon charging and discharging, which further reduces a decrease in the discharge capacity even upon repeated charging and discharging. In this case, gas generation due to the decomposition reaction of the electrolytic solution is also suppressed.

The nitrile compound is a compound including one or more cyano groups (—CN). Specific examples of the nitrile compound include octanenitrile, benzonitrile, phthalonitrile, succinonitrile, glutaronitrile, adiponitrile, cebaconitrile, 1,3,6-hexanetricarbonitrile, 3,3′-oxydipropionitrile, 3-butoxypropionitrile, ethylene glycol bispropionitrile ether, 1,2,2,3-tetracyanopropane, tetracyanopropane, fumaronitrile, 7,7,8,8-tetracyanoquinodimethane, cyclopentanecarbonitrile, 1,3,5-cyclohexanetricarbonitrile, and 1,3-bis(dicyanomethylidene) indane.

Note that the cyanated cyclic carbonic acid ester described above is excluded from the nitrile compound described here.

When manufacturing the electrolytic solution, the electrolyte salt is added to the solvent, following which the triple bond compound and the fluorophosphoric acid salt are added to the solvent. The electrolyte salt, the triple bond compound, and the fluorophosphoric acid salt are thereby each dissolved or dispersed in the solvent. As a result, the electrolytic solution is prepared.

According to the electrolytic solution, the electrolytic solution includes the triple bond compound and the fluorophosphoric acid salt. The triple bond compound includes the first triple bond compound, the second triple bond compound, or both. The fluorophosphoric acid salt includes the first fluorophosphoric acid salt, the second fluorophosphoric acid salt, or both.

In this case, as described above, in the secondary battery including the electrolytic solution, the synergistic action between the triple bond compound and the fluorophosphoric acid salt allows for the formation of the film having superior electrochemical durability on the surface of the negative electrode 22. This suppresses the decomposition reaction of the electrolytic solution on the surface of the negative electrode 22 even upon repeated charging and discharging, which reduces a decrease in the discharge capacity. Accordingly, it is possible to obtain a high discharge capacity even upon repeated charging and discharging. As a result, it is possible to achieve a superior battery characteristic.

In particular, the carbon number of the alkyl group in each of Formulae (1) and (2) regarding the triple bond compound may be within the range from 1 to 7 both inclusive. This improves solubility and compatibility of the triple bond compound. Accordingly, it is possible to achieve higher effects.

Further, the alkali metal element in each of Formulae (3) and (4) regarding the fluorophosphoric acid salt may include lithium. This allows the fluorophosphoric acid salt to sufficiently serve as an electrolyte salt when the secondary battery is a lithium-ion secondary battery. Accordingly, it is possible to achieve higher effects.

Further, the content of the triple bond compound in the electrolytic solution may be within a range from 0.01 vol % to 5 vol % both inclusive. This sufficiently improves the electrochemical durability of the film provided on the surface of the negative electrode 22. Accordingly, it is possible to achieve higher effects.

Further, the content of the fluorophosphoric acid salt in the electrolytic solution may be within a range from 0.01 vol % to 2 vol % both inclusive. This sufficiently improves the electrochemical durability of the film provided on the surface of the negative electrode 22. Accordingly, it is possible to achieve higher effects.

Further, the electrolytic solution may include any one or more of the unsaturated cyclic carbonic acid ester, the fluorinated cyclic carbonic acid ester, or the cyanated cyclic carbonic acid ester. This further suppresses the decomposition reaction of the electrolytic solution. Accordingly, it is possible to achieve higher effects.

Further, the electrolytic solution may include any one or more of the sulfonic acid ester, the sulfuric acid ester, the sulfurous acid ester, the dicarboxylic acid anhydride, the disulfonic acid anhydride, the sulfonic acid carboxylic acid anhydride, or the sulfobenzoic acid imide. This further suppresses the decomposition reaction of the electrolytic solution. Accordingly, it is possible to achieve higher effects.

A description is given next of a secondary battery including the electrolytic solution including described above according to an embodiment.

The secondary battery to be described here is a secondary battery in which a battery capacity is obtained through insertion and extraction of an electrode reactant, and includes a positive electrode, a negative electrode, and an electrolytic solution.

In the secondary battery, a charge capacity of the negative electrode is greater than a discharge capacity of the positive electrode. In other words, an electrochemical capacity per unit area of the negative electrode is set to be greater than an electrochemical capacity per unit area of the positive electrode. This is to prevent precipitation of the electrode reactant on a surface of the negative electrode during charging.

The electrode reactant is not particularly limited in kind, and is specifically a light metal such as an alkali metal or an alkaline earth metal. Examples of the alkali metal include lithium, sodium, and potassium. Examples of the alkaline earth metal include beryllium, magnesium, and calcium.

Examples are given below of a case where the electrode reactant is lithium. A secondary battery in which the battery capacity is obtained through insertion and extraction of lithium is what is called a lithium-ion secondary battery. In the lithium-ion secondary battery, lithium is inserted and extracted in an ionic state.

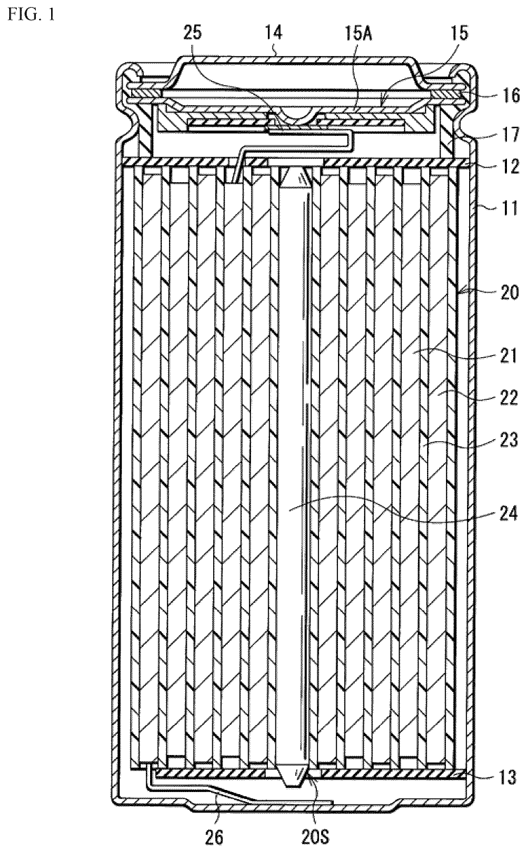

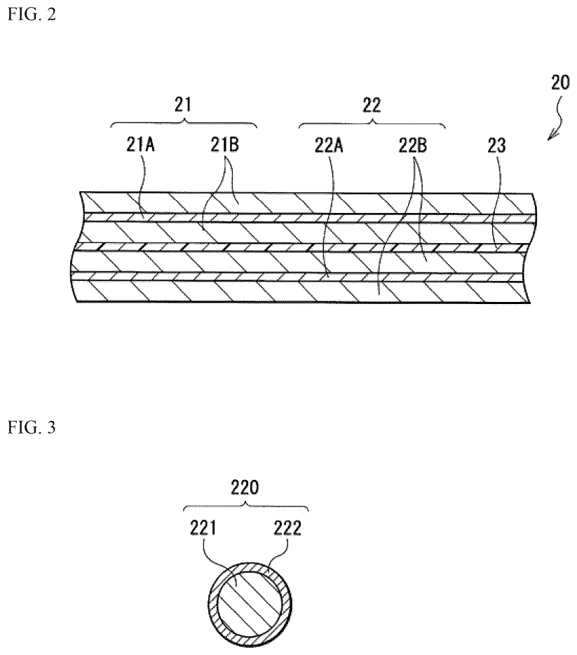

FIG. 1 illustrates a sectional configuration of the secondary battery. FIG. 2 illustrates a sectional configuration of a battery device 20 illustrated in FIG. 1.

As illustrated in FIGS. 1 and 2, the secondary battery mainly includes a battery can 11, a pair of insulating plates 12 and 13, the battery device 20, a positive electrode lead 25, and a negative electrode lead 26. The secondary battery described here is a secondary battery of a cylindrical type in which the battery device 20 is contained inside the battery can 11 having a cylindrical shape.

As illustrated in FIG. 1, the battery can 11 is a container member that contains the battery device 20 and other components. The battery can 11 has one end part that is open and another end part that is closed, and thus has a hollow structure. Further, the battery can 11 includes any one or more of metal materials including, without limitation, iron, aluminum, an iron alloy, and an aluminum alloy. Note that the battery can 11 may have a surface plated with a metal material such as nickel.

A battery cover 14, a safety valve mechanism 15, and a thermosensitive resistive device (a PTC device) 16 are crimped at the open end part of the battery can 11 by means of a gasket 17. The battery can 11 is thereby sealed by the battery cover 14. Here, the battery cover 14 includes a material similar to the material included in the battery can 11. The safety valve mechanism 15 and the PTC device 16 are each disposed on an inner side of the battery cover 14. The safety valve mechanism 15 is electrically coupled to the battery cover 14 via the PTC device 16. The gasket 17 includes an insulating material. The gasket 17 may have a surface on which a material such as asphalt is applied.

When an internal pressure of the battery can 11 reaches a certain level or higher as a result of a cause such as an internal short circuit or heating from outside, a disk plate 15A in the safety valve mechanism 15 inverts, thereby cutting off electrical coupling between the battery cover 14 and the battery device 20. An electric resistance of the PTC device 16 increases in accordance with a rise in temperature, in order to prevent abnormal heat generation resulting from a large current.

As illustrated in FIG. 1, the insulating plates 12 and 13 are so provided as to be opposed to each other with the battery device 20 interposed therebetween. The battery device 20 is thereby sandwiched between the insulating plates 12 and 13.

As illustrated in FIGS. 1 and 2, the battery device 20 is a power generation device that includes a positive electrode 21, a negative electrode 22, a separator 23, and an electrolytic solution (not illustrated).

The battery device 20 is what is called a wound electrode body. That is, the positive electrode 21 and the negative electrode 22 are stacked on each other with the separator 23 interposed therebetween, and are wound, being opposed to each other with the separator 23 interposed therebetween. A center pin 24 is disposed in a winding center space 20S provided at a winding center of the battery device 20. However, the center pin 24 may be omitted.

The positive electrode 21 includes, as illustrated in FIG. 2, a positive electrode current collector 21A and a positive electrode active material layer 21B.

The positive electrode current collector 21A has two opposed surfaces on each of which the positive electrode active material layer 21B is to be provided. The positive electrode current collector 21A includes an electrically conductive material such as a metal material. Specific examples of the electrically conductive material include aluminum.

The positive electrode active material layer 21B includes any one or more of positive electrode active materials into which lithium is insertable and from which lithium is extractable. Note that the positive electrode active material layer 21B may further include any one or more of other materials including, without limitation, a positive electrode binder and a positive electrode conductor. A method of forming the positive electrode active material layer 21B is not particularly limited, and specifically includes a method such as a coating method.

Here, the positive electrode active material layer 21B is provided on each of the two opposed surfaces of the positive electrode current collector 21A. Accordingly, the positive electrode 21 includes two positive electrode active material layers 21. Note that the positive electrode active material layer 21B may be provided only on one of the two opposed surfaces of the positive electrode current collector 21A on a side where the positive electrode 21 is opposed to the negative electrode 22, and the positive electrode 21 may thus include only one positive electrode active material layer 21B.

The positive electrode active material is not particularly limited in kind, and specific examples thereof include a lithium-containing compound. The lithium-containing compound is a compound that includes lithium and one or more transition metal elements as constituent elements. The lithium-containing compound may further include one or more other elements as one or more constituent elements. The one or more other elements are not particularly limited in kind as long as the one or more other elements are each an element other than lithium and the transition metal elements. Specifically, the one or more other elements are any one or more of elements belonging to groups 2 to 15 in the long period periodic table. The lithium-containing compound is not particularly limited in kind, and is specifically, for example, an oxide, a phosphoric acid compound, a silicic acid compound, and a boric acid compound.

Specific examples of the oxide include LiNiO₂, LiCoO₂, LiCo_{0.98}Al_{0.01}Mg_{0.01}O₂, LiNi_{0.5}Co_{0.2}Mn_{0.3}O₂, and LiMn₂O₄. Specific examples of the phosphoric acid compound include LiFePO₄, LiMnPO₄, and LiFe_{0.5}Mn_{0.5}PO₄.

The positive electrode binder includes any one or more of materials including, without limitation, a synthetic rubber and a polymer compound. Specific examples of the synthetic rubber include a styrene-butadiene-based rubber, a fluorine-based rubber, and ethylene propylene diene. Specific examples of the polymer compound include polyvinylidene difluoride, polyimide, and carboxymethyl cellulose.

The positive electrode conductor includes any one or more of electrically conductive materials including, without limitation, a carbon material. Specific examples of the carbon material include graphite, carbon black, acetylene black, and Ketjen black. Note that the electrically conductive material may be a metal material or a polymer compound, for example.

The negative electrode 22 includes, as illustrated in FIG. 2, a negative electrode current collector 22A and a negative electrode active material layer 22B.

The negative electrode current collector 22A has two opposed surfaces on each of which the negative electrode active material layer 22B is to be provided. The negative electrode current collector 22A includes an electrically conductive material such as a metal material. Specific examples of the electrically conductive material include copper.

The negative electrode active material layer 22B includes any one or more of negative electrode active materials into which lithium is insertable and from which lithium is extractable. Note that the negative electrode active material layer 22B may further include any one or more of other materials including, without limitation, a negative electrode binder and a negative electrode conductor. A method of forming the negative electrode active material layer 22B is not particularly limited, and specifically includes any one or more of methods including, without limitation, a coating method, a vapor-phase method, a liquid-phase method, a thermal spraying method, and a firing (sintering) method.

Here, the negative electrode active material layer 22B is provided on each of the two opposed surfaces of the negative electrode current collector 22A. Accordingly, the negative electrode 22 includes two negative electrode active material layers 22B. Note that the negative electrode active material layer 22B may be provided only on one of the two opposed surfaces of the negative electrode current collector 22A on a side where the negative electrode 22 is opposed to the positive electrode 21, and the negative electrode 22 may thus include only one negative electrode active material layer 22B.

The negative electrode active material is not particularly limited in kind, and specific examples thereof include a carbon material and a metal-based material. A reason for this is that a high energy density is obtainable.

Specific examples of the carbon material include graphitizable carbon, non-graphitizable carbon, and graphite (natural graphite and artificial graphite).

The metal-based material is a material including, as one or more constituent elements, any one or more elements among metal elements and metalloid elements that are each able to form an alloy with lithium. Specific examples of such metal elements and metalloid elements include silicon and tin. The metal-based material may be a simple substance, an alloy, a compound, a mixture of two or more thereof, or a material including two or more phases thereof. Specific examples of the metal-based material include TiSi₂ and SiOₓ (0<x≤2 or 0.2<x<1.4).

Details of the negative electrode binder are similar to those of the positive electrode binder. Details of the negative electrode conductor are similar to those of the positive electrode conductor.

The separator 23 is an insulating porous film interposed between the positive electrode 21 and the negative electrode 22 as illustrated in FIG. 2, and allows a lithium ion to pass therethrough while preventing contact (a short circuit) between the positive electrode 21 and the negative electrode 22. The separator 23 includes a polymer compound such as polyethylene.

The positive electrode 21, the negative electrode 22, and the separator 23 are each impregnated with the electrolytic solution, and the electrolytic solution has the configuration described above. That is, the electrolytic solution includes the triple bond compound and the fluorophosphoric acid salt.

As illustrated in FIGS. 1 and 2, the positive electrode lead 25 is coupled to the positive electrode current collector 21A of the positive electrode 21, and includes an electrically conductive material such as aluminum. The positive electrode lead 25 is electrically coupled to the battery cover 14 via the safety valve mechanism 15.

As illustrated in FIGS. 1 and 2, the negative electrode lead 26 is coupled to the negative electrode current collector 22A of the negative electrode 22, and includes an electrically conductive material such as nickel. The negative electrode lead 26 is electrically coupled to the battery can 11.

The secondary battery operates as below.

Upon charging, in the battery device 20, lithium is extracted from the positive electrode 21, and the extracted lithium is inserted into the negative electrode 22 via the electrolytic solution. Upon discharging, in the battery device 20, lithium is extracted from the negative electrode 22, and the extracted lithium is inserted into the positive electrode 21 via the electrolytic solution. Upon the charging and discharging, lithium is inserted and extracted in an ionic state.

When manufacturing the secondary battery, the positive electrode 21 and the negative electrode 22 are fabricated and the electrolytic solution is prepared, following which the secondary battery is assembled using the positive electrode 21, the negative electrode 22, and the electrolytic solution and the assembled secondary battery is subjected to a stabilization process, in accordance with an example procedure described below. Note that a procedure for preparing the electrolytic solution is as described above.

First, a positive electrode mixture is obtained by mixing the positive electrode active material, the positive electrode binder, and the positive electrode conductor with each other, following which the positive electrode mixture is put into a solvent to thereby prepare a positive electrode mixture slurry in paste form. The solvent may be an aqueous solvent, or may be an organic solvent. Thereafter, the positive electrode mixture slurry is applied on the two opposed surfaces of the positive electrode current collector 21A to thereby form the positive electrode active material layers 21B. Thereafter, the positive electrode active material layers 21B may be compression-molded by means of, for example, a roll pressing machine. Lastly, the positive electrode active material layers 21B may be heated. The positive electrode active material layers 21B may be compression-molded multiple times. The positive electrode active material layers 21B are thus formed on the two respective opposed surfaces of the positive electrode current collector 21A. As a result, the positive electrode 21 is fabricated.

The negative electrode 22 is formed by a procedure similar to the fabrication procedure of the positive electrode 21 described above. Specifically, first, a mixture (a negative electrode mixture) in which the negative electrode active material, the negative electrode binder, and the negative electrode conductor are mixed with each other is put into a solvent to thereby prepare a negative electrode mixture slurry in paste form. Thereafter, the negative electrode mixture slurry is applied on the two opposed surfaces of the negative electrode current collector 22A to thereby form the negative electrode active material layers 22B. Thereafter, the negative electrode active material layers 22B may be compression-molded. The negative electrode active material layers 22B are thus formed on the two respective opposed surfaces of the negative electrode current collector 22A. As a result, the negative electrode 22 is fabricated.

First, the positive electrode lead 25 is coupled to the positive electrode current collector 21A of the positive electrode 21 by a joining method such as a welding method, and the negative electrode lead 26 is coupled to the negative electrode current collector 22A of the negative electrode 22 by a joining method such as a welding method. Thereafter, the positive electrode 21 and the negative electrode 22 are stacked on each other with the separator 23 interposed therebetween, following which the stack of the positive electrode 21, the negative electrode 22, and the separator 23 is wound to thereby fabricate a wound body (not illustrated) having the winding center space 20S. The wound body has a configuration similar to that of the battery device 20 except that the positive electrode 21, the negative electrode 22, and the separator 23 are each not impregnated with the electrolytic solution. Thereafter, the center pin 24 is placed in the winding center space 20S of the wound body.

Thereafter, the wound body is sandwiched between the insulating plates 12 and 13, and in that state, the wound body and the insulating plates 12 and 13 are placed inside the battery can 11. In this case, the positive electrode lead 25 is coupled to the safety valve mechanism 15 by a joining method such as a welding method, and the negative electrode lead 26 is coupled to the battery can 11 by a joining method such as a welding method. Thereafter, the electrolytic solution is injected into the battery can 11 to thereby impregnate the wound body with the electrolytic solution. Thus, the positive electrode 21, the negative electrode 22, and the separator 23 are each impregnated with the electrolytic solution, and the battery device 20 is fabricated as a result.

Lastly, the battery cover 14, the safety valve mechanism 15, and the PTC device 16 are placed inside the battery can 11, following which the battery can 11 is crimped by means of the gasket 17. Thus, the battery cover 14, the safety valve mechanism 15, and the PTC device 16 are fixed to the battery can 11, and the battery device 20 is sealed in the battery can 11. As a result, the secondary battery is assembled.

The assembled secondary battery is charged and discharged. Various conditions including, for example, an environment temperature, the number of times of charging and discharging (the number of cycles), and charging and discharging conditions may be set as desired. As a result, a film is formed on the surface of each of the positive electrode 21 and the negative electrode 22, which electrochemically stabilizes a state of the secondary battery. As a result, the secondary battery is completed.

According to the secondary battery, the secondary battery includes the electrolytic solution, and the electrolytic solution has the above-described configuration. In this case, the decomposition reaction of the electrolytic solution on the surface of the negative electrode 22 is suppressed even upon repeated charging and discharging for the reason described above, which reduces a decrease in the discharge capacity. Accordingly, it is possible to achieve a superior battery characteristic.

In particular, the secondary battery may include a lithium-ion secondary battery. This makes it possible to obtain a sufficient battery capacity stably through insertion and extraction of lithium. Accordingly, it is possible to achieve higher effects.

The configuration of the secondary battery described above is appropriately modifiable as described below. Note that any two or more of the following series of modification examples may be combined with each other.

FIG. 3 illustrates a sectional configuration of a secondary battery according toan embodiment. More specifically, FIG. 3 schematically illustrates an enlarged sectional configuration of a negative electrode active material 220 included in the negative electrode 22. The secondary battery described here has a configuration similar to that of the secondary battery already described, except that the negative electrode active material 220 is included.

The negative electrode active material layer 22B includes negative electrode active materials that are each in form of a particle (negative electrode active materials 220), and the negative electrode active materials 220 each include a center part 221 and a covering part 222 as illustrated in FIG. 3. Note that FIG. 3 illustrates only one negative electrode active material 220.

The center part 221 includes any one or more of materials into which lithium is insertable and from which lithium is extractable. The materials include, without limitation, the carbon material described above and the metal-based material described above.

The covering part 222 covers a surface of the center part 221. The covering part 222 may cover the entire surface of the center part 221, or may cover only a portion of the surface of the center part 221. In the latter case, multiple covering parts 222 may cover the surface of the center part 221 at respective locations separate from each other.

The covering part 222 is formed through a stabilization process (initial charging and discharging) on the assembled secondary battery in a manufacturing process of the secondary battery. In the stabilization process, the covering part 222 is so formed as to cover the surface, of the center part 221, having reactivity. Accordingly, the reactivity of the surface of the center part 221 decreases, owing to the covering part 222. Reactivity of a surface of the negative electrode active material 220 thus decreases, which suppresses the decomposition reaction of the electrolytic solution on the surface of the negative electrode active material 220. Accordingly, the decomposition reaction of the electrolytic solution is suppressed also upon subsequent charging and discharging, which electrochemically stabilizes the state of the secondary battery.

Note that in the stabilization process, as described above, in addition to the covering part 222 being formed on the surface of the center part 221, a film may be formed also on a surface of the positive electrode active material in some cases. A reason for this is that the decomposition reaction of the electrolytic solution on the surface of the positive electrode active material is also suppressed.

In particular, the covering part 222 includes nickel as a constituent element. Nickel in the covering part 222 is not particularly limited in form, and may be a simple substance, a compound, an alloy, or a mixture of two or more thereof.

A reason why the covering part 222 includes nickel as a constituent element is that physical strength of the covering part 222 improves, and the covering part 222 is thereby maintained easily even upon repeated charging and discharging. This further suppresses the decomposition reaction of the electrolytic solution, which further reduces a decrease in the discharge capacity even upon repeated charging and discharging.

Note that a method of including nickel as a constituent element in the covering part 222 is not particularly limited. In other words, a source of nickel is not particularly limited.

Specifically, when the positive electrode 21 is used as the source of nickel, the positive electrode active material layer 21B may further include nickel powder. The nickel powder is what is called powdered nickel. A content of the nickel powder in the positive electrode active material layer 21B is not particularly limited, and may be set as desired.

In this case, the positive electrode 21 is fabricated by a similar procedure except that the nickel powder is further added to the positive electrode mixture.

Alternatively, when the electrolytic solution is used as the source of nickel, the electrolytic solution may further include any one or more of nickel compounds. The nickel is a compound including nickel as a constituent element. The nickel compound is not particularly limited in kind, and specific examples thereof include nickel acetate. A content of the nickel compound in the electrolytic solution is not particularly limited, and may be set as desired.

In this case, the electrolytic solution is prepared by a similar procedure except that the nickel compound is further added to the solvent.

The description has been given here of the case where either the positive electrode 21 or the electrolytic solution is used as the source of nickel; however, both the positive electrode 21 and the electrolytic solution may be used as the sources of nickel.

In this case, the covering part 222 includes nickel as a constituent element. Accordingly, the physical strength of the covering part 222 further improves. This suppresses the decomposition reaction of the electrolytic solution even upon repeated charging and discharging, which further reduces a decrease in the discharge capacity. Accordingly, it is possible to achieve higher effects.

The description has been given of the case where the secondary battery has a battery structure of the cylindrical type. However, although not specifically illustrated here, a kind of the battery structure is not particularly limited, and may be, for example, a laminated-film type, a prismatic type, a coin type, or a button type.

The separator 23 that is a porous film is used. However, although not specifically illustrated here, a separator of a stacked type including a polymer compound layer may be used.

Specifically, the separator of the stacked type includes a porous film having two opposed surfaces, and the polymer compound layer provided on one of or each of the two opposed surfaces of the porous film. A reason for this is that adherence of the separator to each of the positive electrode 21 and the negative electrode 22 improves to suppress misalignment (winding displacement) of the battery device 20. This suppresses swelling of the secondary battery even if, for example, the decomposition reaction of the electrolytic solution occurs. The polymer compound layer includes a polymer compound such as polyvinylidene difluoride. A reason for this is that the polymer compound such as polyvinylidene difluoride is superior in physical strength and is electrochemically stable.

Note that the porous film, the polymer compound layer, or both may each include any one or more kinds of insulating particles. A reason for this is that the insulating particles promote heat dissipation upon heat generation by the secondary battery, thus improving safety or heat resistance of the secondary battery. The insulating particles include an inorganic material, a resin material, or both. Specific examples of the inorganic material include aluminum oxide, aluminum nitride, boehmite, silicon oxide, titanium oxide, magnesium oxide, and zirconium oxide. Specific examples of the resin material include acrylic resin and styrene resin.

When fabricating the separator of the stacked type, a precursor solution including, without limitation, the polymer compound and a solvent is prepared, following which the precursor solution is applied on one of or each of the two opposed surfaces of the porous film. In this case, insulating particles may be added to the precursor solution on an as-needed basis.

When the separator of the stacked type is used also, a lithium ion is movable between the positive electrode 21 and the negative electrode 22, and similar effects are therefore achievable. In this case, in particular, the secondary battery improves in safety, as described above. Accordingly, it is possible to achieve higher effects.

The electrolytic solution, which is a liquid electrolyte, is used. However, although not specifically illustrated here, an electrolyte layer, which is a gel electrolyte, may be used.

In the battery device 20 including the electrolyte layer, the positive electrode 21 and the negative electrode 22 are stacked on each other with the separator 23 and the electrolyte layer interposed therebetween, and the stack of the positive electrode 21, the negative electrode 22, the separator 23, and the electrolyte layer is wound. The electrolyte layer is interposed between the positive electrode 21 and the separator 23, and between the negative electrode 22 and the separator 23.

Specifically, the electrolyte layer includes a polymer compound together with the electrolytic solution. The electrolytic solution is held by the polymer compound. A reason for this is that leakage of the electrolytic solution is prevented. The configuration of the electrolytic solution is as described above. The polymer compound includes, for example, polyvinylidene difluoride. When forming the electrolyte layer, a precursor solution including, for example, the electrolytic solution, the polymer compound, and a solvent is prepared, following which the precursor solution is applied on one side or both sides of the positive electrode 21 and on one side or both sides of the negative electrode 22.

When the electrolyte layer is used also, a lithium ion is movable between the positive electrode 21 and the negative electrode 22 via the electrolyte layer, and similar effects are therefore achievable. In this case, in particular, the leakage of the electrolytic solution is prevented, as described above. Accordingly, it is possible to achieve higher effects.

Applications (application examples) of the secondary battery are not particularly limited. The secondary battery used as a power source may serve as a main power source or an auxiliary power source in, for example, electronic equipment and an electric vehicle. The main power source is preferentially used regardless of the presence of any other power source. The auxiliary power source may be used in place of the main power source, and is switched from the main power source.

Specific examples of the applications of the secondary battery include: electronic equipment; apparatuses for data storage; electric power tools; battery packs to be mounted on, for example, electronic equipment; medical electronic equipment; electric vehicles; and electric power storage systems. Examples of the electronic equipment include video cameras, digital still cameras, mobile phones, laptop personal computers, headphone stereos, portable radios, and portable information terminals. Examples of the apparatuses for data storage include backup power sources and memory cards. Examples of the electric power tools include electric drills and electric saws. Examples of the medical electronic equipment include pacemakers and hearing aids. Examples of the electric vehicles include electric automobiles including hybrid automobiles. Examples of the electric power storage systems include battery systems for home use or industrial use in which electric power is accumulated for a situation such as emergency. In each of the above-described applications, one secondary battery may be used, or multiple secondary batteries may be used.

The battery packs may each include a single battery, or may each include an assembled battery. The electric vehicle is a vehicle that operates (travels) with the secondary battery as a driving power source, and may be a hybrid automobile that is additionally provided with a driving source other than the secondary battery. In the electric power storage system for home use, electric power accumulated in the secondary battery that is an electric power storage source may be utilized for using, for example, home appliances.

An application example of the secondary battery will now be described in detail. The configuration of the application example described below is merely an example, and is appropriately modifiable.

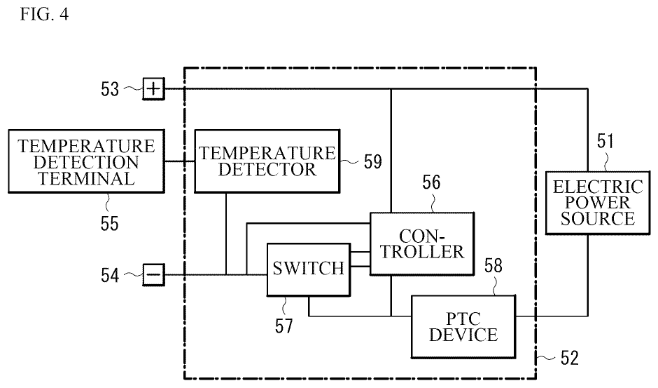

FIG. 4 illustrates a block configuration of a battery pack. The battery pack described here is a battery pack (what is called a soft pack) including one secondary battery, and is to be mounted on, for example, electronic equipment typified by a smartphone.

As illustrated in FIG. 4, the battery pack includes an electric power source 51 and a circuit board 52. The circuit board 52 is coupled to the electric power source 51, and includes a positive electrode terminal 53, a negative electrode terminal 54, and a temperature detection terminal 55.

The electric power source 51 includes one secondary battery. The secondary battery has a positive electrode lead coupled to the positive electrode terminal 53 and a negative electrode lead coupled to the negative electrode terminal 54. The electric power source 51 is couplable to outside via the positive electrode terminal 53 and the negative electrode terminal 54, and is thus chargeable and dischargeable. The circuit board 52 includes a controller 56, a switch 57, a PTC device 58, and a temperature detector 59. However, the PTC device 58 may be omitted.

The controller 56 includes, for example, a central processing unit (CPU) and a memory, and controls an overall operation of the battery pack. The controller 56 detects and controls a use state of the electric power source 51 on an as-needed basis.

If a voltage of the electric power source 51 (the secondary battery) reaches an overcharge detection voltage or an overdischarge detection voltage, the controller 56 turns off the switch 57. This prevents a charging current from flowing into a current path of the electric power source 51. The overcharge detection voltage is not particularly limited, and is specifically 4.20 V+0.05 V. The overdischarge detection voltage is not particularly limited, and is specifically 2.40 V+0.1 V.

The switch 57 includes, for example, a charge control switch, a discharge control switch, a charging diode, and a discharging diode. The switch 57 performs switching between coupling and decoupling between the electric power source 51 and external equipment in accordance with an instruction from the controller 56. The switch 57 includes, for example, a metal-oxide-semiconductor field-effect transistor (MOSFET). The charging and discharging currents are detected based on an ON-resistance of the switch 57.

The temperature detector 59 includes a temperature detection device such as a thermistor. The temperature detector 59 measures a temperature of the electric power source 51 through the temperature detection terminal 55, and outputs a result of the temperature measurement to the controller 56. The result of the temperature measurement to be obtained by the temperature detector 59 is used, for example, when the controller 56 performs charge and discharge control upon abnormal heat generation or when the controller 56 performs a correction process upon calculating a remaining capacity.

### EXAMPLES

A description is given of Examples of the present technology according to an embodiment.

### Experiment Examples 1 to 18 and Comparative Examples 1 to 5

Secondary batteries were manufactured, following which the secondary batteries were each evaluated for a battery characteristic as described below.

### [Manufacturing of Secondary Battery]

The lithium-ion secondary batteries of the cylindrical type illustrated in FIGS. 1 and 2 were manufactured in accordance with the following procedure.

### [Fabrication of Positive Electrode]

First, 91 parts by mass of the positive electrode active material (lithium cobalt oxide (LiCoO₂) as the lithium-containing compound (the oxide)), 3 parts by mass of the positive electrode binder (polyvinylidene difluoride), and 6 parts by mass of the positive electrode conductor (graphite) were mixed with each other to thereby obtain a positive electrode mixture. Thereafter, the positive electrode mixture was put into a solvent (N-methyl-2-pyrrolidone as the organic solvent), following which the solvent was stirred to thereby prepare a positive electrode mixture slurry in paste form. Thereafter, the positive electrode mixture slurry was applied on the two opposed surfaces of the positive electrode current collector 21A (a band-shaped aluminum foil having a thickness of 12 μm) by means of a coating apparatus, following which the applied positive electrode mixture slurry was dried to thereby form the positive electrode active material layers 21B. Lastly, the positive electrode active material layers 21B were compression-molded by means of a roll pressing machine. In this manner, the positive electrode 21 was fabricated.

### [Fabrication of Negative Electrode]

First, 93 parts by mass of the negative electrode active material and 7 parts by mass of the negative electrode binder (polyvinylidene difluoride) were mixed with each other to thereby obtain a negative electrode mixture. Used as the negative electrode active material was a mixture of 63 parts by mass of the carbon material (artificial graphite) and 30 parts by mass of the metal-based material (silicon oxide (SiO)). Thereafter, the negative electrode mixture was put into a solvent (N-methyl-2-pyrrolidone as the organic solvent), following which the solvent was stirred to thereby prepare a negative electrode mixture slurry in paste form. Thereafter, the negative electrode mixture slurry was applied on the two opposed surfaces of the negative electrode current collector 22A (a band-shaped copper foil having a thickness of 15 μm) by means of a coating apparatus, following which the applied negative electrode mixture slurry was dried to thereby form the negative electrode active material layers 22B. Lastly, the negative electrode active material layers 22B were compression-molded by means of a roll pressing machine. In this manner, the negative electrode 22 was fabricated.

### [Preparation of Electrolytic Solution]

A solvent (ethylene carbonate as the cyclic carbonic acid ester and dimethyl carbonate as the chain carbonic acid ester) was prepared. A mixture ratio (a weight ratio) between ethylene carbonate and dimethyl carbonate in the solvent was set to 20:80. Thereafter, the electrolyte salt (lithium hexafluorophosphate (LiPF₆) as the lithium salt) was added to the solvent, following which the solvent was stirred. The content of the electrolyte salt was set to 1.2 mol/kg with respect to the solvent. Lastly, the triple bond compound and the fluorophosphoric acid salt were added to the solvent to which the electrolyte salt was added, following which the solvent was stirred. A classification, a kind, and a content (wt %) of the triple bond compound and a classification, a kind, and a content (wt %) of the fluorophosphoric acid salt were as listed in Table 1. As a result, the electrolytic solution was prepared.

Specifically, used as the first triple bond compound were methyl 2-octynoate (OCM), methyl 2-nonynoate (NNM), methyl 2-hexynoate (HXM), methyl 2-heptynoate (HPM), ethyl 2-pentynoate (PNE), ethyl 2-butynoate (BTE), and methyl 2-decynoate (DCM). Used as the second triple bond compound was diisopropyl 2-butynedioate (BTDI).

Used as the first fluorophosphoric acid salt were lithium difluorophosphate (DFPL) and sodium difluorophosphate (DFPN). Used as the second fluorophosphoric acid salt was dilithium monofluorophosphate (MFPL).

As indicated in Table 2, an electrolytic solution for comparison was prepared by a similar procedure except that either the triple bond compound or the fluorophosphoric acid was not used or neither the triple bond compound nor the fluorophosphoric acid was used.

“Classification” presented in Tables 1 and 2 indicates as below. In “Classification” regarding the triple bond compound, “First” indicates the first triple bond compound, and “Second” indicates the second triple bond compound. In “Classification” regarding the fluorophosphoric acid salt, “First” indicates the first fluorophosphoric acid salt, and “Second” indicates the second fluorophosphoric acid salt.

### [Assembly of Secondary Battery]

First, the positive electrode lead 25 (an aluminum foil) was welded to the positive electrode current collector 21A of the positive electrode 21, and the negative electrode lead 26 (a copper foil) was welded to the negative electrode current collector 22A of the negative electrode 22.

Thereafter, the positive electrode 21 and the negative electrode 22 were stacked on each other with the separator 23 (a fine porous polyethylene film having a thickness of 15 μm) interposed therebetween, following which the stack of the positive electrode 21, the negative electrode 22, and the separator 23 was wound to thereby fabricate the wound body having the winding center space 20S. Thereafter, the center pin 24 was placed in the winding center space 20S of the wound body.

Thereafter, the wound body was placed inside the battery can 11 together with the insulating plates 12 and 13. In this case, the positive electrode lead 25 was welded to the safety valve mechanism 15, and the negative electrode lead 26 was welded to the battery can 11. Thereafter, the electrolytic solution was injected into the battery can 11. The wound body was thereby impregnated with the electrolytic solution, and the battery device 20 was thus fabricated.

Lastly, the battery cover 14, the safety valve mechanism 15, and the PTC device 16 were placed inside the battery can 11, following which the battery can 11 was crimped by means of the gasket 17. Thus, the battery can 11 was sealed. As a result, the secondary battery was assembled.

### [Stabilization of Secondary Battery]

The secondary battery was charged and discharged for one cycle in an ambient temperature environment (at a temperature of 23° C.). Upon charging, the secondary battery was charged with a constant current of 0.1 C until a voltage reached 4.2 V, and was thereafter charged with a constant voltage of that value, 4.2 V, until a current reached 0.05 C. Upon discharging, the secondary battery was discharged with a constant current of 0.1 C until the voltage reached 3.0 V. Note that 0.1 C was a value of a current that caused a battery capacity (a theoretical capacity) to be completely discharged in 10 hours, and 0.05 C was a value of a current that caused the battery capacity to be completely discharged in 20 hours. As a result, the secondary battery was completed.

After the completion of the secondary battery, the content (wt %) of the triple bond compound in the electrolytic solution and the content (wt %) of the fluorophosphoric acid salt in the electrolytic solution were measured by ICP optical emission spectroscopy. The results of the measurement were as presented in Tables 1 and 2.

### [Evaluation of Battery Characteristic]

The secondary batteries were each evaluated for a cyclability characteristic as the battery characteristic in accordance with the following procedure, and the evaluation revealed the results presented in Tables 1 and 2.

First, the secondary battery was charged in a high-temperature environment (at a temperature of 50° C.), following which the charged secondary battery was left standing (for a standing time of 3 hours) in the same environment. Upon charging, the secondary battery was charged with a constant current of 1 C until a voltage reached 4.2 V, and was thereafter charged with a constant voltage of that value, 4.2 V, until a current reached 0.05 C. Note that 1 C was a value of a current that caused the battery capacity to be completely discharged in 1 hour.

Thereafter, the secondary battery was discharged in the same environment to thereby measure a discharge capacity (a first-cycle discharge capacity). Upon discharging, the secondary battery was discharged with a constant current of 3 C until the voltage reached 3.0 V. Note that 3 C was a value of a current that caused the battery capacity to be completely discharged in ⅓ hours. Thereafter, the secondary battery was repeatedly charged and discharged in the same environment until the number of cycles reached 100 to thereby measure the discharge capacity (a 100th-cycle discharge capacity). Charging and discharging conditions of the second and subsequent cycles were similar to the charging and discharging conditions of the first cycle.

Lastly, a capacity retention rate that was an index for evaluating the cyclability characteristic was calculated based on the following calculation expression: capacity retention rate

$$( \% ) = ( 100 th - cycle discharge capacity / first - cycle discharge capacity ) \times 100.$$

TABLE 1

| | | | | | | | Capacity |
|---|---|---|---|---|---|---|---|
| | Triple bond compound | | | Fluorophosphoric acid salt | | | retention |
| | | | Content | | | Content | rate |
| | Classification | Kind | (wt %) | Classification | Kind | (wt %) | (%) |
| Example 1 | First | OCM | 0.01 | First | DFPL | 0.2 | 64 |
| Example 2 | First | OCM | 0.1 | First | DFPL | 0.2 | 65 |
| Example 3 | First | OCM | 1 | First | DFPL | 0.2 | 67 |
| Example 4 | First | OCM | 5 | First | DFPL | 0.2 | 61 |
| Example 5 | First | OCM | 7 | First | DFPL | 0.2 | 55 |
| Example 6 | First | OCM | 1 | First | DFPL | 0.01 | 65 |
| Example 7 | First | OCM | 1 | First | DFPL | 0.5 | 68 |
| Example 8 | First | OCM | 1 | First | DFPL | 2 | 64 |
| Example 9 | First | OCM | 1 | First | DFPL | 5 | 57 |
| Example 10 | First | NNM | 1 | First | DFPL | 0.2 | 66 |
| Example 11 | First | HXM | 1 | First | DFPL | 0.2 | 66 |
| Example 12 | First | HPM | 1 | First | DFPL | 0.2 | 67 |
| Example 13 | First | PNE | 1 | First | DFPL | 0.2 | 66 |
| Example 14 | First | BTE | 1 | First | DFPL | 0.2 | 67 |
| Example 15 | First | DCM | 1 | First | DFPL | 0.2 | 65 |

TABLE 2

| | | | | | | | Capacity |
|---|---|---|---|---|---|---|---|
| | Triple bond compound | | | Fluorophosphoric acid salt | | | retention |
| | | | Content | | | Content | rate |
| | Classification | Kind | (wt %) | Classification | Kind | (wt %) | (%) |
| Example 16 | Second | BTDI | 1 | First | DFPL | 0.2 | 68 |
| Example 17 | First | OCM | 1 | First | DFPN | 0.2 | 65 |
| Example 18 | First | OCM | 1 | Second | MFPL | 0.2 | 65 |
| Comparative | — | — | — | — | — | — | 42 |
| example 1 | | | | | | | |
| Comparative | First | OCM | 1 | — | — | — | 43 |
| example 2 | | | | | | | |
| Comparative | Second | BTDI | 1 | — | — | — | 43 |
| example 3 | | | | | | | |
| Comparative | — | — | — | First | DFPL | 0.2 | 45 |
| example 4 | | | | | | | |
| Comparative | — | — | — | Second | MFPL | 0.2 | 43 |
| example 5 | | | | | | | |

As indicated in Tables 1 and 2, the capacity retention rate varied depending on the configuration of the electrolytic solution.

In the following, the capacity retention rate when the electrolytic solution included neither the triple bond compound nor the fluorophosphoric acid salt (Comparative example 1) was used as a comparison reference.

When the electrolytic solution included only the triple bond compound (Comparative examples 2 and 3), the capacity retention rate slightly increased. Further, when the electrolytic solution included only the fluorophosphoric acid salt (Comparative examples 4 and 5) also, the capacity retention rate slightly increased.

It was expected from these results (Comparative examples 1 to 5) that, even if the electrolytic solution included both the triple bond compound and the fluorophosphoric acid salt, the capacity retention rate would increase only slightly.

In fact, however, a result different from the expectation described above was obtained. That is, when the electrolytic solution included both the triple bond compound and the fluorophosphoric acid salt (Examples 1 to 18), the capacity retention rate markedly increased.

A possible reason why the capacity retention rate markedly increased was that the synergistic action between the triple bond compound and the fluorophosphoric acid salt allowed for the formation of the film having superior electrochemical durability on the surface of the negative electrode 22.

In particular, when the electrolytic solution included both the triple bond compound and the fluorophosphoric acid salt (Examples 1 to 18), the following series of tendencies was obtained.

Firstly, both when the first triple bond compound was used as the triple bond compound and when the second triple bond compound was used as the triple bond compound a high capacity retention rate was obtained. Secondly, both when the first fluorophosphoric acid salt was used as the fluorophosphoric acid salt and when the second fluorophosphoric acid salt was used as the fluorophosphoric acid salt, a high capacity retention rate was obtained. Thirdly, when the content of the triple bond compound in the electrolytic solution was within the range from 0.01 wt % to 5 wt % both inclusive, the capacity retention rate further increased. Fourthly, when the content of the fluorophosphoric acid salt in the electrolytic solution was within the range from 0.01 wt % to 2 wt % both inclusive, the capacity retention rate further increased.

### Examples 19 to 24

Secondary batteries were fabricated by a procedure similar to that in Example 3, except that the electrolytic solution included the additive (the unsaturated cyclic carbonic acid ester, the fluorinated cyclic carbonic acid ester, or the cyanated cyclic carbonic acid ester) as indicated in Table 3, following which the secondary batteries were each evaluated for a battery characteristic. A classification, a kind, and a content (wt %) of the additive were as listed in Table 3.

Specifically, used as the unsaturated cyclic carbonic acid ester was vinylene carbonate (VC). Used as the fluorinated cyclic carbonic acid ester was fluoroethylene carbonate (FEC). Used as the cyanated cyclic carbonic acid ester was cyanoethylene carbonate (CEC).

TABLE 3

| | | | | | | | | | | Capacity |
|---|---|---|---|---|---|---|---|---|---|---|
| | Triple bond compound | | | Fluorophosphoric acid salt | | | Additive | | | retention |
| | | | Content | | | Content | | | Content | rate |
| | Classification | Kind | (wt %) | Classification | Kind | (wt %) | Classification | Kind | (wt %) | (%) |
| Example 19 | First | OCM | 1 | First | DFPL | 0.2 | Unsaturated cyclic | VC | 1 | 75 |
| Example 20 | | | | | | | carbonic acid ester | | 5 | 76 |
| Example 21 | | | | | | | Fluorinated cyclic | FEC | 1 | 76 |
| Example 22 | | | | | | | carbonic acid ester | | 5 | 78 |
| Example 23 | | | | | | | Cyanated cyclic | CEC | 1 | 77 |
| Example 24 | | | | | | | carbonic acid ester | | 5 | 78 |

As indicated in Table 3, when the electrolytic solution included the additive (the unsaturated cyclic carbonic acid ester, the fluorinated cyclic carbonic acid ester, or the cyanated cyclic carbonic acid ester) (Examples 19 to 24), the capacity retention rate further increased, as compared with when the electrolytic solution included no additive (Example 3).

### Examples 25 to 44

Secondary batteries were fabricated by a procedure similar to that in Example 3, except that the electrolytic solution included the additive (the sulfonic acid ester, the sulfuric acid ester, the sulfurous acid ester, the dicarboxylic acid anhydride, the disulfonic acid anhydride, the sulfonic acid carboxylic acid anhydride, or the sulfobenzoic acid imide) as indicated in Tables 4 and 5, following which the secondary batteries were each evaluated for a battery characteristic. The classification, the kind, and the content (wt %) of the additive were as listed in Tables 4 and 5.

Specifically, used as the sulfonic acid ester were 1,3-propane sultone (PS), 1-propene-1,3-sultone (PRS), 1,4-butane sultone (BS1), 2,4-butane sultone (BS2), and methanesulfonate propargyl ester (MSP).

Used as the sulfuric acid ester were 1,3,2-dioxathiolane 2,2-dioxide (OTO), 1,3,2-dioxathiane 2,2-dioxide (OTA), and 4-methylsulfonyloxymethyl-2,2-dioxo-1,3,2-dioxathiolane (SOTO).

Used as the sulfurous acid ester were 1,3,2-dioxathiolane 2-oxide (DTO) and 4-methyl-1,3,2-dioxathiolane 2-oxide (MDTO).

Used as the dicarboxylic acid anhydride were 1,4-dioxane-2,6-dione (DOD), succinic anhydride (SA), and glutaric anhydride (GA).

Used as the disulfonic acid anhydride were 1,2-ethanedisulfonic anhydride (ESA), 1,3-propanedisulfonic anhydride (PSA), and hexafluoro 1,3-propanedisulfonic anhydride (FPSA).

Used as the sulfonic acid carboxylic acid anhydride were 2-sulfobenzoic anhydride (SBA) and 2,2-dioxooxathiolane-5-one (DOTO).

Used as the sulfobenzoic acid imide were o-sulfobenzimide (SBI) and N-methylsaccharin (NMS).

TABLE 4

| | | | | | | | | | | Capacity |
|---|---|---|---|---|---|---|---|---|---|---|
| | Triple bond compound | | | Fluorophosphoric acid salt | | | Additive | | | retention |
| | | | Content | | | Content | | | Content | rate |
| | Classification | Kind | (wt %) | Classification | Kind | (wt %) | Classification | Kind | (wt %) | (%) |
| Example 25 | First | OCM | 1 | First | DFPL | 0.2 | Sulfonic | PS | 1 | 76 |
| Example 26 | | | | | | | acid ester | PRS | 1 | 74 |
| Example 27 | | | | | | | | BS1 | 1 | 73 |
| Example 28 | | | | | | | | BS2 | 1 | 73 |
| Example 29 | | | | | | | | MSP | 1 | 77 |
| Example 30 | | | | | | | Sulfuric | OTO | 1 | 75 |
| Example 31 | | | | | | | acid ester | OTA | 1 | 74 |
| Example 32 | | | | | | | | SOTO | 1 | 75 |
| Example 33 | | | | | | | Sulfurous | DTO | 1 | 76 |
| Example 34 | | | | | | | acid ester | MDTO | 1 | 75 |
| Example 35 | | | | | | | Dicarboxylic | DOD | 1 | 73 |
| Example 36 | | | | | | | acid | SA | 1 | 73 |
| Example 37 | | | | | | | anhydride | GA | 1 | 74 |

TABLE 5

| | | | | | | | | | | Capacity |
|---|---|---|---|---|---|---|---|---|---|---|
| | Triple bond compound | | | Fluorophosphoric acid salt | | | Additive | | | retention |
| | | | Content | | | Content | | | Content | rate |
| | Classification | Kind | (wt %) | Classification | Kind | (wt %) | Classification | Kind | (wt %) | (%) |
| Example 38 | First | OCM | 1 | First | DFPL | 0.2 | Disulfonic | ESA | 1 | 78 |
| Example 39 | | | | | | | acid | PSA | 1 | 79 |
| Example 40 | | | | | | | anhydride | FPSA | 1 | 75 |
| Example 41 | | | | | | | Sulfonic acid | SBA | 1 | 75 |
| Example 42 | | | | | | | carboxylic | DOTO | 1 | 76 |
| | | | | | | | acid anhydride | | | |
| Example 43 | | | | | | | Sulphobenzoic | SBI | 1 | 73 |
| Example 44 | | | | | | | acid imide | NMS | 1 | 74 |

As indicated in Tables 4 and 5, when the electrolytic solution included the additive (the sulfonic acid ester, the sulfuric acid ester, the sulfurons acid ester, the dicarboxylic acid anhydride, the disulfonic acid anhydride, the sulfonic acid carboxylic acid anhydride, or the sulfobenzoic acid imide) (Examples 25 to 44), the capacity retention rate further increased, as compared with when the electrolytic solution included no additive (Example 3). [Examples 45 and 46]

Secondary batteries were fabricated by a procedure similar to that in Example 3, except that the negative electrode active material 220 (the center part 221 and the covering part 222) was used as the negative electrode active material as indicated in Table 6, following which the secondary batteries were each evaluated for a battery characteristic.

When the positive electrode 21 was used as the source of nickel, the positive electrode 21 was fabricated by a procedure of a similar procedure except that nickel powder (having a median diameter D50 of 0.2 μm) was added to the positive electrode mixture. In this case, a portion of the positive electrode conductor was replaced with the nickel powder, and a content of the nickel powder in the positive electrode mixture was set to 0.01 parts by mass.

When the electrolytic solution was used as the source of nickel, the electrolytic solution was prepared by a procedure of a similar procedure except that a nickel compound (nickel acetate·tetrahydrate) was further added to the solvent to which the triple bond compound and the fluorophosphoric acid salt were added. In this case, a content of the nickel compound in the electrolytic solution was set to 1 wt %.

The negative electrode 22 was fabricated by a similar procedure except that the center part 221 (artificial graphite) was used instead of the negative electrode active material (artificial graphite). The covering part 222 including nickel as a constituent element was thus formed on the surface of the center part 221 through a stabilization process on the assembled secondary battery. Accordingly, the negative electrode active material 220 including the center part 221 and the covering part 222 was formed. As a result, the negative electrode 22 including the negative electrode active material 220 was fabricated.

After the completion of the secondary battery, the secondary battery was disassembled to thereby collect the negative electrode active material 220. Thereafter, the negative electrode active material 220 was analyzed by a scanning electron microscope (a scanning electron microscope SU3800/SU3900 available from High-Tech Corporation), an energy dispersive X-ray spectrometer (EDS), and an X-ray photoelectron spectrometer (EDX). Results of the analysis of the negative electrode active material 220 were as listed in Table 6.

TABLE 6

| | | | | | | | Covering part | | Capacity |
|---|---|---|---|---|---|---|---|---|---|
| | Triple bond compound | | | Fluorophosphoric acid salt | | | Presence | | retention |
| | | | Content | | | Content | or absence | Source of | rate |
| | Classification | Kind | (wt %) | Classification | Kind | (wt %) | of nickel | nickel | (%) |
| Example 45 | First | OCM | 1 | First | DFPL | 0.2 | Present | Positive electrode | 74 |
| Example 46 | | | | | | | Present | Electrolytic solution | 74 |

As indicated in Table 6, when the negative electrode active material 220 including the center part 221 and the covering part 222 was used (Examples 45 and 46), the capacity retention rate further increased, as compared with when the negative electrode active material 220 was not used (Example 3).

Based upon the results presented in Tables 1 to 6, when the electrolytic solution included the triple bond compound and the fluorophosphoric acid salt, a high capacity retention rate was obtained. Therefore, the cyclability characteristic improved. Accordingly, it was possible to achieve a superior battery characteristic of the secondary battery including the electrolytic solution.

Although the present technology has been described above with reference to some embodiments and Examples, the configuration of the present technology is not limited to those described with reference to the embodiments and Examples above, and is therefore modifiable in a variety of ways.

Specifically, the description has been given of the case where the battery device has a device structure of a wound type. However, the device structure of the battery device is not particularly limited, and may be of any other type such as a stacked type or a zigzag folded type. In the stacked type, the positive electrode and the negative electrode are alternately stacked on each other with the separator interposed therebetween. In the zigzag folded type, the positive electrode and the negative electrode are opposed to each other with the separator interposed therebetween, and are folded in a zigzag manner.

Further, although the description has been given of the case where the electrode reactant is lithium, the electrode reactant is not particularly limited. Specifically, the electrode reactant may be another alkali metal such as sodium or potassium, or may be an alkaline earth metal such as beryllium, magnesium, or calcium, as described above. In addition, the electrode reactant may be another light metal such as aluminum.

The effects described herein are mere examples, and effects of the present technology are therefore not limited to those described herein. Accordingly, the present technology may achieve any other effect.

Note that the present technology may have the following configurations according to an embodiment.

<1>

A secondary battery including:
a positive electrode;
a negative electrode; and
an electrolytic solution including a triple bond compound and a fluorophosphoric acid salt, in which
the triple bond compound includes a compound represented by Formula (1), a compound represented by Formula (2), or both, and
the fluorophosphoric acid salt includes a compound represented by Formula (3), a compound represented by Formula (4), or both,

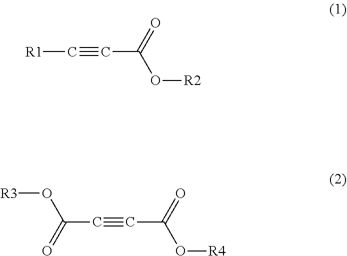

where each of R1 to R4 is an alkyl group,

M1PF₂O₂ (3)
where M1 is an alkali metal element,

M2₂PFO₃ (4)
where M2 is an alkali metal element.
   <2>

The secondary battery according to <1>, in which carbon number of the alkyl group is greater than or equal to 1 and less than or equal to 7.

<3>

The secondary battery according to <1> or <2>, in which the alkali metal element includes lithium.

<4>

The secondary battery according to any one of <1> to <3>, in which a content of the triple bond compound in the electrolytic solution is greater than or equal to 0.01 wt % and less than or equal to 5 wt %.

<5>

The secondary battery according to any one of <1> to <4>, in which a content of the fluorophosphoric acid salt in the electrolytic solution is greater than or equal to 0.01 wt % and less than or equal to 2 wt %.

<6>

The secondary battery according to any one of <1> to <5>, in which the electrolytic solution further includes at least one of an unsaturated cyclic carbonic acid ester, a fluorinated cyclic carbonic acid ester, or a cyanated cyclic carbonic acid ester.

<7>

The secondary battery according to any one of <1> to <6>, in which the electrolytic solution further includes at least one of a sulfonic acid ester, a sulfuric acid ester, a sulfurous acid ester, a dicarboxylic acid anhydride, a disulfonic acid anhydride, a sulfonic acid carboxylic acid anhydride, or a sulfobenzoic acid imide.

<8>

The secondary battery according to any one of <1> to <7>, in which
the negative electrode includes a negative electrode active material, and
the negative electrode active material includes
   a center part into which an electrode reactant is to be inserted and from which the electrode reactant is to be extracted, and
   a covering part covering a surface of the center part and including nickel as a constituent element.
      <9>

The secondary battery according to any one of <1> to <8>, in which the secondary battery includes a lithium-ion secondary battery.

<10>

An electrolytic solution for a secondary battery, the electrolytic solution including:
a triple bond compound; and
a fluorophosphoric acid salt, in which
the triple bond compound includes a compound represented by Formula (1), a compound represented by Formula (2), or both, and
the fluorophosphoric acid salt includes a compound represented by Formula (3), a compound represented by Formula (4), or both,

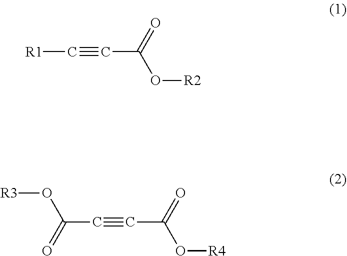

where each of R1 to R4 is an alkyl group,

M1PF₂O₂ (3)
where M1 is an alkali metal element,

M22PFO₃ (4)
where M2 is an alkali metal element.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A secondary battery comprising:
a positive electrode;
a negative electrode; and
an electrolytic solution including a triple bond compound and a fluorophosphoric acid salt, wherein
the triple bond compound includes a compound represented by Formula (1), a compound represented by Formula (2), or both, and
the fluorophosphoric acid salt includes a compound represented by Formula (3), a compound represented by Formula (4), or both, 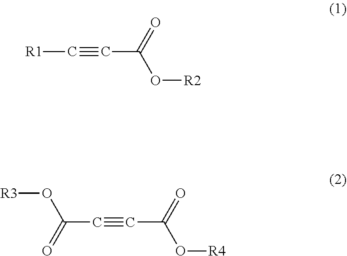
where each of R1 to R4 is an alkyl group,
M1PF₂O₂ (3)
where M1 is an alkali metal element,
M2₂PFO₃ (4)
where M2 is an alkali metal element.

2. The secondary battery according to claim 1, wherein a carbon number of the alkyl group is greater than or equal to 1 and less than or equal to 7.

3. The secondary battery according to claim 1, wherein the alkali metal element comprises lithium.

4. The secondary battery according to claim 1, wherein a content of the triple bond compound in the electrolytic solution is greater than or equal to 0.01 weight percent and less than or equal to 5 weight percent.

5. The secondary battery according to claim 1, wherein a content of the fluorophosphoric acid salt in the electrolytic solution is greater than or equal to 0.01 weight percent and less than or equal to 2 weight percent.

6. The secondary battery according to claim 1, wherein the electrolytic solution further includes at least one of an unsaturated cyclic carbonic acid ester, a fluorinated cyclic carbonic acid ester, or a cyanated cyclic carbonic acid ester.

7. The secondary battery according to claim 1, wherein the electrolytic solution further includes at least one of a sulfonic acid ester, a sulfuric acid ester, a sulfurous acid ester, a dicarboxylic acid anhydride, a disulfonic acid anhydride, a sulfonic acid carboxylic acid anhydride, or a sulfobenzoic acid imide.

8. The secondary battery according to claim 1, wherein
the negative electrode includes a negative electrode active material,
the negative electrode active material includes
a center part into which an electrode reactant is to be inserted and from which the electrode reactant is to be extracted, and
a covering part covering a surface of the center part, and
the covering part includes nickel as a constituent element.

9. The secondary battery according to claim 1, wherein the secondary battery comprises a lithium-ion secondary battery.

10. An electrolytic solution for a secondary battery, the electrolytic solution comprising:
a triple bond compound; and
a fluorophosphoric acid salt, wherein
the triple bond compound includes a compound represented by Formula (1), a compound represented by Formula (2), or both, and
the fluorophosphoric acid salt includes a compound represented by Formula (3), a compound represented by Formula (4), or both, 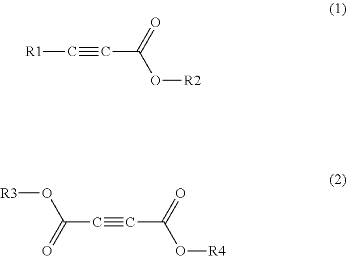
where each of R1 to R4 is an alkyl group,
M1PF₂O₂ (3)
where M1 is an alkali metal element,
M2₂PFO₃ (4)
where M2 is an alkali metal element.
